(19) United States
(12) Patent Application Publication
(10) Pub. No.: US 2009/0281388 A1
(43) Pub. Date: Nov. 12, 2009

(54) ENDOSCOPE COVER AND COVER-TYPE ENDOSCOPE

(75) Inventor: Yoshiaki Ito, Fuchu-shi (JP)

Correspondence Address: Frishauf, Holtz Goodman & Chick, P.C.;16th Floor, 220 Fifth Avenue, New York, NY, 10001-7708, US

(73) Assignee: Olympus Corporation, Tokyo (JP)

(21) Appl. No.: 12/435,507

(22) Filed: May 5, 2009

(30) Foreign Application Priority Data
May 9, 2008 (JP) 2008-124005

(51) Int. Cl. A61B 1/00

(52) U.S. Cl. 600/121

(57) ABSTRACT

An endoscope cover, which is detachably fitted on an insertion section of an endoscope, includes a sheath main body support member to which a proximal end of a sheath section of the endoscope cover is connected, and a built-in tube support member to which a built-in tube that is disposed in the sheath section is connected. The built-in tube support member is coupled to the sheath main body support member in a manner to be movable relative to the sheath main body support member in a longitudinal axial direction of the sheath section.

### CROSS-REFERENCE TO RELATED APPLICATIONS

This application is based upon and claims the benefit of priority from prior Japanese Patent Application No. 2008-124005, filed May 9, 2008, the entire contents of which are incorporated herein by reference.

### BACKGROUND OF THE INVENTION

1. Field of the Invention

The present invention relates to a separation-type endoscope cover which covers an insertion section of an endoscope, and a cover-type endoscope having this endoscope cover.

2. Description of the Related Art

Japanese Patent No. 2787471 (patent document 1), for instance, discloses a cover-type endoscope in which an insertion section of the endoscope is covered with a separable endoscope cover. In this type of cover-type endoscope, an insertion section of the endoscope is inserted in the endoscope cover, and thus the endoscope is assembled in a usable state. In the endoscope cover, tubes (also referred to as built-in tubes or inner tubes), such as a channel tube, an air feed tube and a water feed tube, are disposed with a relatively high density. These tubes receive pushing force and pulling force in the axial direction when the insertion section of the endoscope is bent or curved. Thus, in usual cases, the tubes are disposed in the endoscope cover with loose tensile force so as to be freely movable with allowance.

### BRIEF SUMMARY OF THE INVENTION

According to an aspect of the present invention, there is provided an endoscope cover which is detachably fitted on an insertion section of an endoscope, comprising: a cylindrical sheath main body which is detachably fitted on the insertion section of the endoscope; an operation section coupling member which is provided at a proximal end portion of the sheath main body and is capable of being coupled to an operation section of the endoscope; and a built-in tube which is disposed in the sheath main body and has a distal end attached to a distal end portion of the sheath main body, wherein the operation section coupling member includes a sheath main body support member to which a proximal end of the sheath main body is connected, and a built-in tube support member to which a proximal side portion of the built-in tube is connected, and the built-in tube support member is coupled to the sheath main body support member in a manner to be movable relative to the sheath main body support member in a longitudinal axial direction of the sheath main body.

Preferably, the operation section coupling member is provided one of the sheath main body support member and the built-in tube support member is provided with a cylindrical slide portion which extends in the longitudinal axial direction of the sheath main body, the other of the sheath main body support member and the built-in tube support member is provided with a cylindrical slide reception portion which is slidably fitted on the slide portion, and the slide portion and the slide reception portion are relatively movable in the longitudinal axial direction, thereby to adjust slack in the built-in tube.

Preferably, the operation section coupling member is provided an elastic member, which elastically urges the built-in tube support member toward the operation section side, relative to the sheath main body support member, is provided between the sheath main body support member and the built-in tube support member.

Preferably, the built-in tube support member includes an inlet for insertion of the insertion section of the endoscope, and a guide portion which restricts a direction of insertion of the insertion section of the endoscope, which is inserted from the inlet into the endoscope cover, and guides the insertion section of the endoscope to a position where slack in the built-in tube is eliminated.

According to another aspect of the invention, there is provided a cover-type endoscope in which an endoscope cover is detachably fitted on an insertion section of the endoscope, the endoscope cover comprising: a cylindrical sheath main body which is detachably fitted on the insertion section of the endoscope; an operation section coupling member which is provided at a proximal end portion of the sheath main body and is capable of being coupled to an operation section of the endoscope; and a built-in tube which is disposed in the sheath main body and has a distal end attached to a distal end portion of the sheath main body, wherein the operation section coupling member includes a sheath main body support member to which a proximal end of the sheath main body is connected, and a built-in tube support member to which a proximal side portion of the built-in tube is connected, and the built-in tube support member is coupled to the sheath main body support member in a manner to be movable relative to the sheath main body support member in a longitudinal axial direction of the sheath main body.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1A is a perspective view showing a main body section of a cover-type endoscope according to a first embodiment of the present invention;

FIG. 1B is a perspective view showing a sheath section of the cover-type endoscope according to the first embodiment;

FIG. 2 is a perspective view showing the cover-type endoscope according to the first embodiment, which is in the coupled state;

FIG. 3A is a perspective view showing, in enlarged scale, the main body section of the cover-type endoscope according to the first embodiment;

FIG. 3B is a perspective view showing, in enlarged scale, the sheath section of the cover-type endoscope according to the first embodiment;

FIG. 4 is a vertical cross-sectional view of a bending section and inner tubes of the cover-type endoscope according to the first embodiment;

FIG. 5 is an exploded perspective view showing a proximal end portion of the sheath section of the cover-type endoscope according to the first embodiment;

FIG. 6 is an exploded perspective view showing a coupling part of the cover-type endoscope according to the first embodiment;

FIG. 7 is a partial cross-sectional rear view showing the proximal end portion of the sheath section of the cover-type endoscope according to the first embodiment;

FIG. 8A is a cross-sectional view taken along line VIII-VIII in FIG. 7, showing a slide contact part between a slide reception part of a tube coupling reception portion and a slide part of a sheath coupling reception portion of the sheath section of the cover-type endoscope according to the first embodiment;

FIG. 8B is a cross-sectional view taken along line VIII-VIII in FIG. 7, showing the state in which a proximal end of the sheath coupling reception portion abuts on a stopper surface of a block portion of the sheath section of the cover-type endoscope according to the first embodiment; and

FIG. 9 is a longitudinal cross-sectional view showing a proximal end portion of a sheath section of a cover-type endoscope according to a second embodiment of the present invention.

### DETAILED DESCRIPTION OF THE INVENTION

The present invention relates to an endoscope which is called a cover-separation-type endoscope or a cover-type endoscope, wherein an insertion section of the endoscope is covered with a separation-type endoscope cover. An endoscope according to a first embodiment of the present invention will now be described with reference to the accompanying drawings.

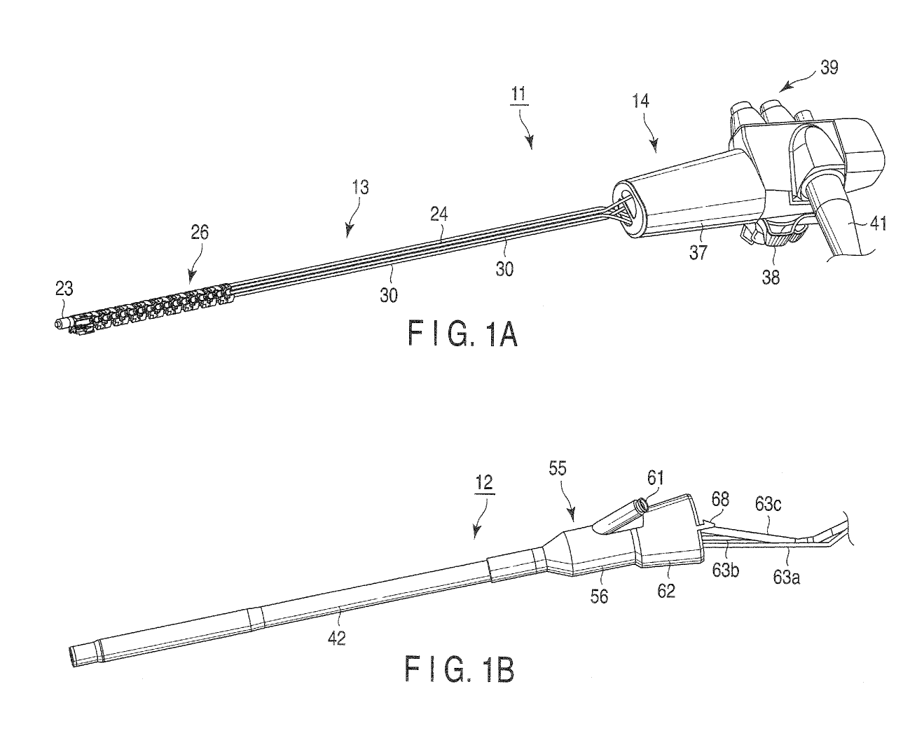

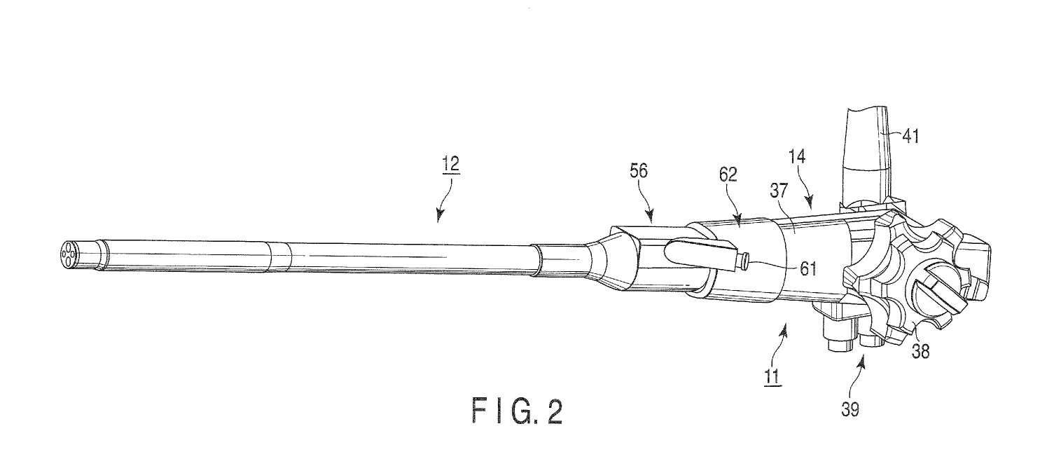

The cover-type endoscope comprises a main body section 11 shown in FIG. 1A and a sheath section 12 shown in FIG. 1B. FIG. 2 shows the state in which the sheath section 12 is detachably assembled to the main body section 11. The main body section 11 is a reuse section which is repeatedly used, and the sheath section 12 is a disposable section which is disposed of as a separable endoscope cover.

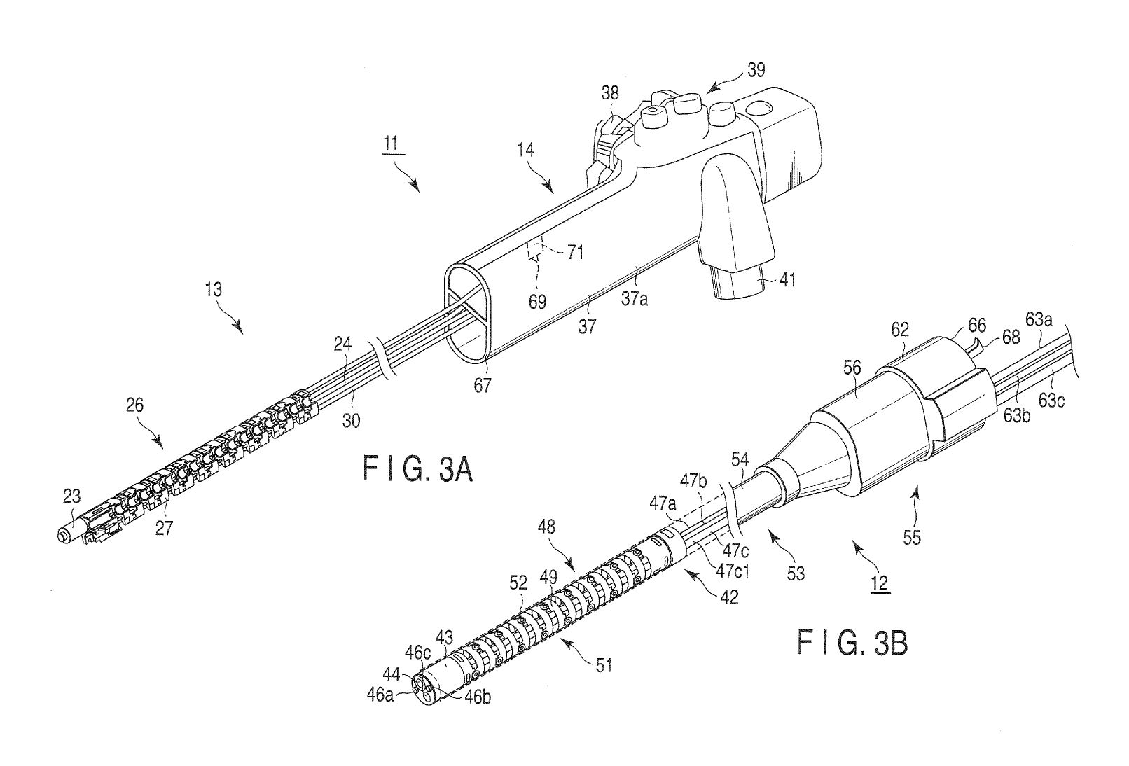

As shown in FIG. 1A and FIG. 3A, the main body section 11 comprises a built-in section (insertion section) 13 and an operation section 14. The built-in section 13 is inserted in the sheath section 12. An observation unit 23 is disposed at a distal end of the built-in section 13. The observation unit 23 is configured to include an illumination unit which illuminates an object of observation, and an image pickup unit which captures an observation image. A distal end portion of an electric cable 24 is connected to the observation unit 23. A proximal end portion of the electric cable 24 extends to the operation section 14 side. The electric cable 24 is configured to include various signal lines for transmitting a driving signal which drives the observation unit 23, and an image signal which is obtained by the observation unit.

As shown in FIG. 3A, a bending section 26 is continuously provided on the proximal end side of the observation unit 23. In the bending section 26, a great number of columnar node rings 27, each being short in the longitudinal axial direction of the built-in section 13, are coaxially disposed in line. Further, neighboring ones of the node rings 27 are rotatably coupled along axes crossing the longitudinal axial direction. Thereby, the bending section 26, which is bendable, is constituted.

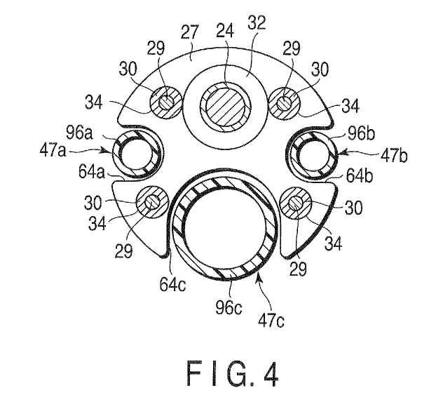

Distal end portions of operation wires 29 (see FIG. 4) for bending the bending section 26 are coupled to the foremost node ring 27. As shown in FIG. 4, the operation wires 29 are inserted in tubular wire guides 30 in the state in which the operations wires 29 are advancible/retreatable. The operation wires 29 and wire guides 30 extend to the proximal end side of the built-in section 13, and are led into the operation section 14.

As shown in FIG. 4, in each node ring 27, one cable insertion hole 32 and four wire guide insertion holes 34 are formed in a manner to penetrate in the longitudinal axial direction. The electric cable 24 is passed through the cable insertion hole 32, and the wire guides 30 are engaged with and inserted in the wire guide insertion holes 34. Each of the electric cable 24 and wire guides 30 extends through the bending section 26 toward the proximal end side of the built-in section 13, and further extends from the proximal end portion of the bending section 26 into the operation section 14.

In the present embodiment, the observation unit 23, the bending section 26 including plural node rings 27, the electric cable 24, the operation wires 29 and the wire guides 30 constitute the built-in section (insertion section) 13. The built-in section 13 is inserted in the sheath section 12 shown in FIG. 3B, and is attached as shown in FIG. 2.

As shown in FIG. 3A, the operation section 14 of the main body section 11 has an operation section housing 37 which forms an outer shell thereof. The operation section housing 37 has a longitudinally elongated oval shape or a substantially rectangular shape. The operation section housing 37 has a hold portion 37a which can be grasped. The hold portion 37a is formed of a portion of the operation section housing 37, which is relatively elongated from the distal end side to the proximal end side of the operation section housing 37. A bending operation knob 38 is provided on one side surface of a proximal end portion of the operation section housing 37. By rotating the bending operation knob 38, the above-described operation wires 29 are advanced/retreated. The operation of bending the bending section 26 can be performed by the advancing/retreating operation wires 29.

Various operation switches 39 for observation, air feeding and water feeding are provided on a top surface of the proximal end portion of the operation section housing 37. A universal cord 41 extends from the other side surface of the proximal end portion of the operation section housing 37. The above-described electric cable 24 and signal lines from the operation switches 39 are passed through the universal cord 41. The universal cord 41 is connected to a video processor for an observation operation, an air/water feed device for air/water feed operations, and a suction device for a suction operation, which are not shown.

Next, referring to FIGS. 3A and 3B, the structure of the cover-separation-type endoscope is described in detail. The sheath section 12 includes an insertion sheath section 42 serving as a sheath main body in which the built-in section 13 is inserted. A sheath distal end portion 43 is provided at the distal end of the insertion sheath section 42. A distal end surface portion of the sheath distal end portion 43 is provided with an observation window 44. When the built-in section 13 is inserted in the sheath section 12, the observation unit 23 is positioned to be opposed to the proximal end side (inner surface) of the observation window 44. The observation unit 23 performs observation in a body cavity through the observation window 44.

An air feed nozzle 46a, a water feed nozzle 46b and a channel opening 46c are formed at a distal end surface of the sheath distal end portion 43. The air feed nozzle 46a and water feed nozzle 46b have nozzle ports which are directed to the observation window 44. The channel opening 46c is an opening for projecting a therapeutic device, and serves also as a suction port. The sheath distal end portion 43 constitutes a distal-end support member which supports the observation window 44, air feed nozzle 46a, channel opening 46c and water feed nozzle 46b. The observation window 44, air feed nozzle 46a, channel opening 46c and water feed nozzle 46b are disposed substantially equidistant in the circumferential direction about the center axis of the sheath section 12.

As shown in FIG. 3B, the insertion sheath section 42 is provided with a sheath bending section 48 which is provided continuous with the proximal end side of the sheath distal end portion 43. In the sheath bending section 48, a plurality of circular-cylindrical sheath node rings 49 are rotatably coupled. The outer periphery of a sheath node ring group, which is a coupled body of the plural sheath node rings 49, is covered with a soft rubber tube 52. A bending tube 51 is formed of the sheath node ring group, which is the coupled body of the sheath node rings 49, and the rubber tube 52.

A flexible tube section 53 is provided continuous with a proximal end of the sheath bending section 48. The flexible tube section 53 is formed of an elongated, flexible tube body 54. In the present embodiment, the sheath main body of the insertion sheath section 42 is formed of the bending tube 51 and the tube body 54. In the meantime, the bending tube 51 may be formed as a tube body portion without using a node ring.

A proximal section 55 is coupled to a proximal end portion of the flexible tube section 53. The proximal section 55 is an operation section coupling member which is connectable to the operation section 14 of the endoscope. Although the concrete structure of the proximal section 55 will be described later, the proximal section 55 comprises a sheath coupling reception portion 56 and a tube coupling reception portion 62. The sheath coupling reception portion 56 is a sheath main body support member which is connected to the proximal end portion of the insertion sheath section 42. The tube coupling reception portion 62 is a built-in tube support member which is movably coupled to the sheath coupling reception portion 56, and to which built-in tubes are connected.

In the above-described sheath distal end portion 43, a distal end portion of an air feed inner tube 47a is coupled to an inner end portion of the air feed nozzle 46a. A distal end portion of a water feed inner tube 47b is coupled to an inner end portion of the water feed nozzle 46b. A distal end portion of a channel inner tube 47c is coupled to an inner end portion of the channel opening 46c.

The inner tubes 47a, 47b and 47c are disposed in the insertion sheath section 42 in a predetermined arrangement state shown in FIG. 4. The distal ends of the inner tubes 47a, 47b and 47c are coupled to the sheath distal end portion 43. The inner tubes 47a, 47b and 47c extend, in the predetermined arrangement state, through the inside of the bending tube 51 and tube body 54, and are led into the proximal section 55.

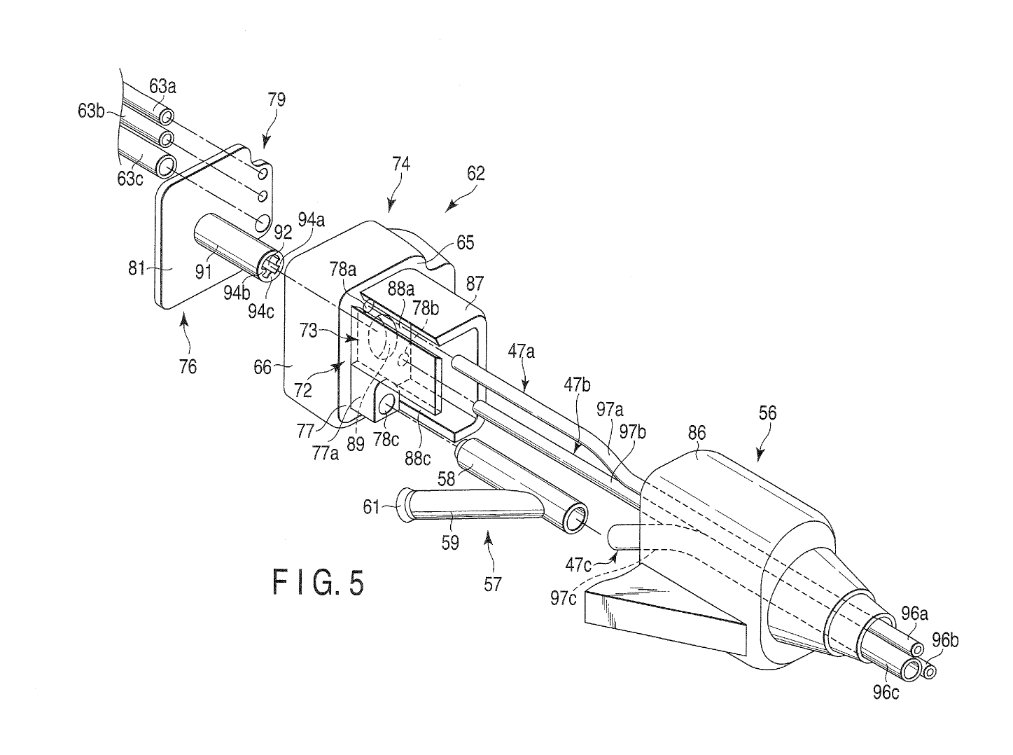

As is shown in FIG. 5, a branch tube portion 57, which is connected to a proximal end portion of the channel inner tube 47c, is provided in the sheath coupling reception portion 56. A proximal end side of the branch tube portion 57 is branched into a suction tube portion 58 and a therapeutic device insertion tube portion 59. The suction tube portion 58 extends to the proximal end side in the longitudinal axial direction of the sheath section 12, and substantially forms the proximal end portion of the channel inner tube 47c.

The therapeutic device insertion tube portion 59 extends obliquely to the proximal end side from the outer peripheral surface of the suction tube portion 58, and forms a therapeutic device insertion hole 61. The therapeutic device insertion hole 61 is so disposed as to be positioned on the same side as the side on which the bending operation knob 38 is provided on the operation section housing 37 of the main body section 11 in the case where the sheath section 12 is attached to the main body section 11.

One end of each of an air feed outer tube 63a, a water feed outer tube 63b and a channel outer tube 63c is connected to the tube coupling reception portion 62. The air feed outer tube 63a is made to communicate with the above-described air feed inner tube 74a. Similarly, the water feed outer tube 63b is made to communication with the water feed inner tube 74b, and the channel outer tube 63c is made to communicate with the channel inner tube 47c. The other ends of the air feed outer tube 63a and water feed outer tube 63b are connected to the air/water feed device (not shown). The other end of the channel outer tube 63c is connected to the suction device (not shown).

Next, a description is given of the insertion section in which the built-in section 13 of the main body section 11 is inserted in the bending tube 51 and tube body 54 of the sheath section 12. As shown in FIG. 4, three tube reception portions (air feed tube reception portion 64a, water feed tube reception portion 64b and channel tube reception portion 64c), which have radially inwardly recessed shapes, are formed in an outer peripheral part of each node ring 27 of the bending section 26 of the built-in section 13.

The relative positional relationship between the air feed tube reception portion 64a, water feed tube reception portion 64b and channel tube reception portion 64c in a cross section perpendicular to the center axis of the bending section 26 (main body section 11) is set to be similar to the relative positional relationship between the inner end portions of the air feed nozzle 46a, water feed nozzle 46b and channel opening 46c in a cross section perpendicular to the center axis of the sheath section 12, as described above. As shown in FIG. 4, in each of the node rings 27, the cable insertion hole 32, in which the electric cable 24 is inserted, the air feed tube reception portion 64a, the channel tube reception portion 64c and the water feed tube reception portion 64b are disposed substantially equidistant in the circumferential direction about the center axis of the bending section 26 (main body section 11).

In the case where the built-in section 13 is inserted in and drawn out of the sheath section 12, the air feed inner tube 47a, water feed inner tube 47b and channel inner tube 47c are inserted and engaged in the air feed tube reception portion 64a, water feed tube reception portion 64b and channel tube reception portion 64c, respectively. By maintaining this relationship of engagement, the inner tubes 47a, 47b and 47c are guided in the insertion/drawing-out direction while the relative positions thereof is restricted. At this time, since the air feed inner tube 47a, water feed inner tube 47b and channel inner tube 47c are accommodated in the associated air feed tube reception portion 64a, water feed tube reception portion 64b and channel tube reception portion 64c, compact assembly is realized.

Next, a description is given of an attachment/detachment mechanism for attaching/detaching the sheath section 12 to/from the main body section 11. As shown in FIG. 3B, a cylindrical attachment reception portion 66, which extends in the longitudinal axial direction of the sheath section 12, is formed on a proximal-end-side part of the tube coupling reception portion 62 of the sheath section 12. On the other hand, as shown in FIG. 3A, an attachment portion 67, which extends in the longitudinal axial direction of the main body section 11, is formed on a distal-end-side part of the operation section 14 of the main body section 11. The attachment portion 67 of the operation section 14 is configured to be removably engaged with, and detachably coupled to, the attachment reception portion 66 of the tube coupling reception portion 62.

A hook-shaped engagement portion 68 is provided at a rear end of the tube coupling reception portion 62. On the other hand, an engagement reception portion 69 for engagement with the engagement portion 68 is provided at a distal end portion of the operation section 14. In the state in which the attachment portion 67 is inserted in the attachment reception portion 66 and is set in the correct attachment position, the engagement portion 68 engages the engagement reception portion 69 and the sheath section 12 is connected to the main body section 11 in the locked state.

As shown in FIG. 3A, a separation button 71 for releasing the engagement between the engagement portion 68 and the engagement reception portion 69 is provided at a distal end portion of the operation section 14. By operating the separation button 71, the engagement of the engagement portion 68 with the engagement reception portion 69 is released. Thereby, the lock state of the sheath section 12 to the main body section 11 can be released.

Next, a description is given of functional sections as a flow path displacement section, an alignment mechanism and an introducing mechanism, which are assembled in the sheath coupling reception portion 56 and the tube coupling reception portion 62. To begin with, FIG. 5 to FIG. 7 are referred to.

The tube coupling reception portion 62 comprises an outer part 74 and an inner part 76 which is mounted in the outer part 74. The outer part 74 includes a thick-plate-like block portion 77 which is perpendicular to the longitudinal axis of the sheath section 12. In the block portion 77, as shown in FIG. 5, an air feed connection port 78a and a water feed connection port 78b, which serve as inner connection portions, are formed in a manner to penetrate in the longitudinal axial direction of the sheath section 12. In the block portion 77, a channel base portion 77a which projects in the longitudinal axial direction is formed at a position corresponding to a channel connection port 78c. In the channel base portion 77a, the channel connection port 78c is formed to penetrate from a distal end surface of the channel base portion 77a in the longitudinal axial direction of the sheath section 12.

Proximal end portions of the air feed inner tube 47a, water feed inner tube 47b and channel inner tube 47c, which are introduced into the sheath coupling reception portion 56, are fixedly connected, by means of, e.g. adhesion, to distal end portions of the air feed connection port 78a, water feed connection port 78b and channel connection port 78c. Thereby, an inner tube connection portion 73 for connection of the inner tubes 47a, 47b and 47c is formed on the distal end side of the tube coupling reception portion 62. The tube coupling reception portion 62 serves as a built-in tube support member for connection of proximal end portions of the respective built-in tubes.

As shown in FIG. 5, the air feed connection port 78a, water feed connection port 78b and channel connection port 78c are disposed along the circumferential direction about the center axis of the sheath section 12, and are disposed in a discrete fashion at radially outside positions. Thereby, the air feed connection port 78a, water feed connection port 78b and channel connection port 78c are disposed at positions corresponding to the positions of the respective inner tubes 47a, 47b and 47c which are connected to the air feed nozzle 46a, water feed nozzle 46b and channel opening 46c in the sheath distal end portion 43.

On the other hand, as shown in FIG. 5, the block portion 77 of the outer part 74 is provided with a cylindrical attachment reception portion 66. The attachment reception portion 66 is formed on the outer part 74 so as to integrally extend to the proximal end side in the longitudinal axial direction. The attachment reception portion 66 has an inner cavity with a rectangular shape in a cross section perpendicular to the longitudinal axis of the sheath section 12.

An outer tube connection portion 79 for connection of the respective outer tubes 63a, 63b and 63c is provided at a proximal-end-side part of the tube coupling reception portion 62. The outer tube connection portion 79 is provided on the attachment reception portion 66.

The inner part 76 includes a plate-like portion 81 having a shape corresponding to the shape of the inner cavity of the attachment reception portion 66. The plate-like portion 81 is configured to be fitted in the attachment reception portion 66. The plate-like portion 81 is fitted in the attachment reception portion 66 from the proximal end side of the attachment reception portion 66. At this time, a distal end surface of the plate-like portion 81 is coupled to a proximal end surface of the block portion 77. Thereby, the outer tube connection portion 79, which is disposed in the space for the outer tubes, is formed on the plate-like portion 81.

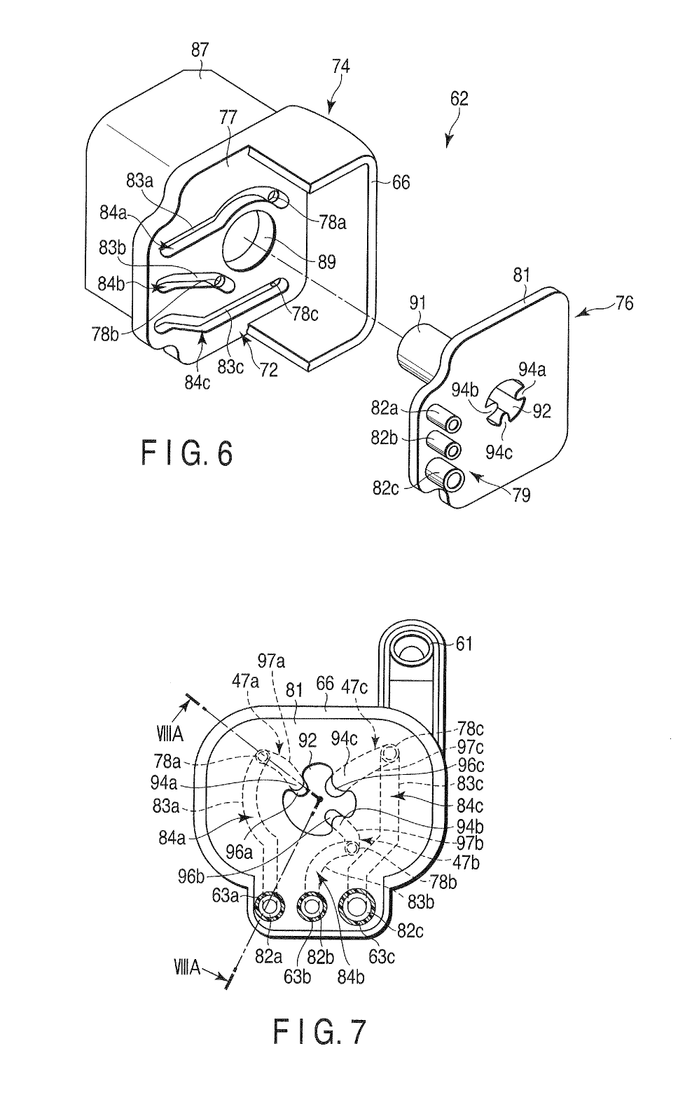

As shown in FIG. 6, the plate-like portion 81 of the outer tube connection portion 79 is provided with an air feed mouthpiece 82a, a water feed mouthpiece 82b and a channel mouthpiece 82c. Distal end portions of the air feed outer tube 63a, water feed outer tube 63b and channel outer tube 63c are connected to the air feed mouthpiece 82a, water feed mouthpiece 82b and channel mouthpiece 82c, respectively. The respective outer tubes 63a, 63b and 63c extend from the proximal end opening of the attachment reception potion 66 to the proximal end side via the space for outer tubes. Further, a substantially circular-cylindrical insertion portion 91, which serves as an insertion hole portion (to be described later), is formed on the plate-like portion 81.

In the case where the sheath section 12 is attached to the main body section 11 as shown in FIG. 2, the air feed mouthpiece 82a, water feed mouthpiece 82b and channel mouthpiece 82c, which are shown in FIG. 6, are disposed on the side where the universal cord 41 is disposed. At this time, the air feed mouthpiece 82a, water feed mouthpiece 82b and channel mouthpiece 82c are directed to the proximal end side and are vertically juxtaposed on the lateral outside of a side surface of the operation section housing 37.

As shown in FIG. 3B, the respective outer tubes 63a, 63b and 63c are vertically juxtaposed on the lateral outside of the side surface of the operation section housing 37, and are made to extend together from the tube coupling reception portion 62 toward the proximal end side. The led-out outer tubes 63a, 63b and 63c are supported on the operation section 14 in the state in which the outer tubes 63a, 63b and 63c are bundled at the proximal end portion of the operation section 14.

As shown in FIG. 6, an air feed groove 83a, a water feed groove 83b and a channel groove 83c are formed in a proximal end surface of the block portion 77 of the outer part 74. The air feed groove 83a establishes communication between the air feed connection port 78a and air feed mouthpiece 82a. The water feed groove 83b establishes communication between the water feed connection port 78b and water feed mouthpiece 82b. The channel groove 83c establishes communication between the channel connection port 78c and channel mouthpiece 82c.

By coupling the plate-like portion 81 to the proximal end surface of the block portion 77, the plate-like portion 81 covers the air feed groove 83a, water feed groove 83b and channel groove 83c of the block portion 77. At this time, an air feed flow path 84a, a water feed flow path 84b and a channel flow path 84c are formed between the proximal end surface of the block portion 77 of the outer part 74 and the plate-like portion 81 of the inner part 76. The air feed flow path 84a connects the air feed connection port 78a and the associated air feed mouthpiece 82a. The water feed flow path 84b connects the water feed connection port 78b and the associated water feed mouthpiece 82b. The channel flow path 84c connects the channel connection port 78c and the associated channel mouthpiece 82c.

Thereby, a manifold portion 72 serving as a flow path displacement section is composed of the air feed flow path 84a, water feed flow path 84b and channel flow path 84c between the plate-like portion 81 and the proximal end surface of the block portion 77. The manifold portion 72 displaces the connection positions of the proximal end portions of the inner tubes 47a, 47b and 47c and the connection positions of the distal end portions of the outer tubes 63a, 63b and 63c to different positions.

Next, a description is given of a slack adjusting mechanism which adjusts the slack of a built-in tube. As shown in FIG. 5, a cylindrical slide portion 86, which extends in the longitudinal axial direction of the sheath section 12, is formed on the proximal end portion of the sheath coupling reception portion 56. On the other hand, a cylindrical slide reception portion 87 is formed on the outer part 74 of the tube coupling reception portion 62. The slide reception portion 87 extends from the distal end portion of the block portion 77 toward the sheath section 12 in the longitudinal axial direction of the sheath section 12.

The slide portion 86 is fitted on the outer periphery of the slide reception portion 87, and the tube coupling reception portion 62 and sheath coupling reception portion 56 are assembled so as to be relatively movable in the longitudinal axial direction of the sheath section 12. At this time, the tube coupling reception portion 62 is movable relative to the sheath coupling reception portion 56 (“relative movement”) in a direction away from, or in a direction toward, the sheath coupling reception portion 56 in the longitudinal axial direction of the sheath section 12. By the movement operation between the tube coupling reception portion 62 and the sheath coupling reception portion 56, the slack in the built-in tubes in the sheath section 12 can be adjusted.

As shown in FIG. 5, the outside diameter of the slide reception portion 87 of the tube coupling reception portion 62 is less than the outside diameter of the block portion 77. Thus, the proximal end of the sheath coupling reception portion 56 abuts on the distal end surface of the block portion 77. Specifically, the distal end surface of the block portion 77 forms a stopper surface 65 upon which the sheath coupling reception portion 56 abuts. The tube coupling reception portion 62 is movable in the longitudinal axial direction of the sheath section 12 over a stroke S, with the stopper surface 65 being set as a push-side terminal end.

In the case where the tube coupling reception portion 62 is pulled to the proximal side, relative to the sheath coupling reception portion 56, each built-in tube is pulled to the proximal side, and a tensile force can be applied to each built-in tube. A pull position (pull-side terminal end), at which an optimal tensile force can be applied, can be understood by the operation by the feeling of force, on the basis of a sharp increase of the resistive force when the tube coupling reception portion 62 is pulled to the proximal side. Alternatively, the pull-side terminal end position may be restricted and the pull position may be determined, for example, by providing indices on the sheath coupling reception portion 56 and the tube coupling reception portion 62 and providing a stopper mechanism such as a click-type stopper mechanism.

As shown in FIG. 5, the vertical cross-sectional shape of each of the slide portion 86 and slide reception portion 87 is not a perfect circular shape but is rectangular. Thus, the slide portion 86 and slide reception portion 87 do not rotate about the axis, and are movable only in the longitudinal axial direction of the sheath section 12. In the case where each of the slide portion 86 and slide reception portion 87 is formed to have a perfect circular cross-sectional shape, a rotation prevention mechanism, such as a key, for preventing rotation about the axis, may be provided.

As shown in FIG. 5, the respective connection ports 78a, 78b and 78c of the outer part 74 of the tube coupling reception portion 62 are disposed with a bias to the center axis from the position of the slide reception portion 87. In addition, in the slide reception portion 87, an air feed tube slit 88a and a branch tube portion slit 88c are properly formed so as to extend in the longitudinal axial direction. The air feed tube slit 88a passes and escapes the air feed inner tube 47a. The branch tube portion slit 88c passes and escapes the branch tube portion 57 of the channel inner tube 47c.

The respective inner tubes 47a, 47b and 47c are introduced into the sheath coupling reception portion 56 from the tube body 54 of the sheath section 12, and then the inner tubes 47a, 47b and 47c flare radially outward and extend to the proximal end side. Further, the inner tubes 47a, 47b and 47c are introduced into the region within the slide reception portion 87 and are connected to the associated connection ports 18a, 78b and 78c.

In the sheath coupling reception portion 56, the parts of the air feed inner tube 47a, water feed inner tube 47b and channel inner tube 47c are displaced outward relative to the center axis of the sheath section 12. Thereby, an air feed led-out portion 97a, a water feed led-out portion 97b and a channel led-out portion 97c are formed of the outwardly displaced parts of the respective inner tubes 47a, 47b and 47c.

According to the above-described structure, if the tube coupling reception portion 62 is slid to the proximal end side (proximal side) relative to the sheath coupling reception portion 56, the inner tubes 47a, 47b and 47c are pulled to the proximal end side. At this time, since the inner tubes 47a, 47b and 47c are pulled to the proximal end side, if there is slack in each inner tube, 47a, 47b, 47c, the slack can be eliminated. In addition, a tensile force acts on each inner tube, 47a, 47b, 47c, and a force for keeping straight extension in the tensile direction can be applied. Accordingly, by retreating the tube coupling reception portion 62 to the proximal end side (proximal side), the slack in each inner tube, 47a, 47b, 47c, can be eliminated, and at the same time, with the application of tensile force, the respective inner tubes 47a, 47b and 47c can be aligned at predetermined positions. Specifically, as shown in FIG. 4, the air feed inner tube 47a, water feed inner tube 47b and channel inner tube 47c are disposed in the bending tube 51 and tube body 54 at predetermined positions in the cross section perpendicular to the center axis of the sheath section 12, and an air feed extension portion 96a, a water feed extension portion 96b and a channel extension portion 96c, which extend straight along the center axis of the sheath section 12, are formed.

The air feed led-out portion 97a, a water feed led-out portion 97b and a channel led-out portion 97c of the air feed inner tube 47a, water feed inner tube 47b and channel inner tube 47c are disposed in the inner space of the slide reception portion 87, and are disposed on the outside of the insertion portion 91 of the plate-like portion 81 of the inner part 76.

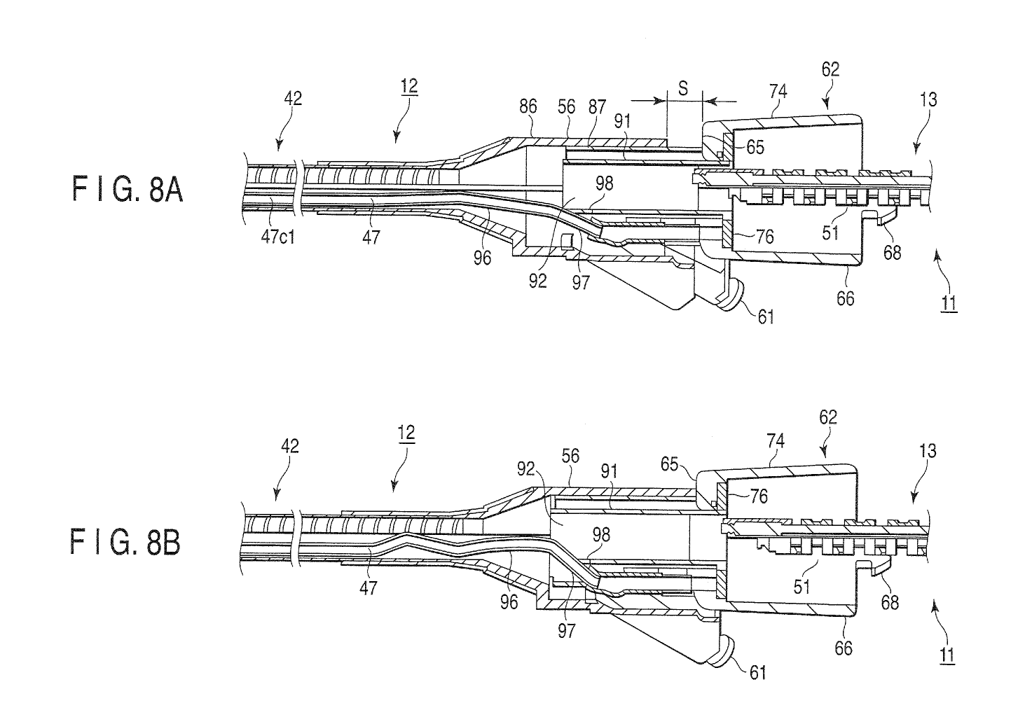

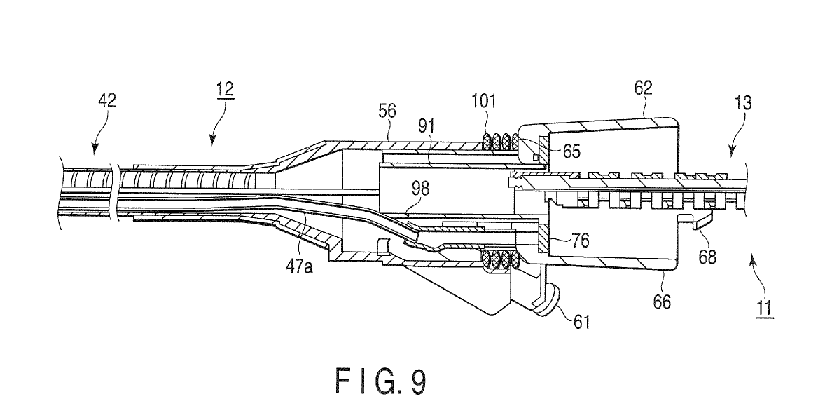

The insertion portion 91 is formed at a central part of the plate-like portion 81 of the inner part 76 so as to extend to the distal end side along the center axis of the inner part 76. As shown in FIG. 8A, the insertion portion 91 is inserted and assembled in an insertion portion introducing hole 89 of the block portion 77, and is disposed in the slide reception portion 87 in a manner to project from the distal end surface of the block portion 77.

The distal end surface of the insertion portion 91 is disposed to be opposed to the proximal end surface of the tube body 54. An insertion hole 92 for passing and guiding the built-in section 13 is formed of the inner cavity of the insertion portion 91. Specifically, the insertion portion 91 serves as a guide section (introducing mechanism) for guiding the built-in section 13 of the main body section 11 into the tube body 54 of the sheath section 12.

As shown in FIG. 8A and FIG. 8B, notch portions (lead-out positioning portions) 98, which engage the air feed led-out portion 97a, water feed led-out portion 97b and channel led-out portion 97c of the three inner tubes 47a, 47b and 47c, are formed at an outlet part of the insertion portion 91. The led-out portions 97a, 97b and 97c may be engaged in these notch portions 98 and the lead-out positions may be restricted. Thereby, the positions of the led-out portions 97a, 97b and 97c of the inner tubes 47a, 47b and 47c, relative to the insertion portion 91, are determined. As a result, as will be described later, the guide function for guiding the built-in section 13 can be enhanced, in combination with the guide function of the insertion portion 91 at the time of introducing the built-in section 13.

The inside diameter of the insertion hole 92 of the insertion portion 91 is such a diameter that the built-in section 13 can be introduced into the insertion hole 92. In this example, in particular, the insertion hole 92 of the insertion portion 91 has an inner peripheral surface with a diameter which is substantially equal to the outside diameter of the node ring 27 of the bending section 26. As shown in FIG. 7, a projection-like air feed guide portion 94a, a projection-like water feed guide portion 94b and a projection-like channel guide portion 94c are formed on the inner peripheral surface of the insertion hole 92 of the insertion portion 91. These guide portions 94a, 94b and 94c extend in ridge shapes over the entire length in the center axis direction. The outer shapes of the air feed guide portion 94a, water feed guide portion 94b and channel guide portion 94c on their projection end side, that is, on the center axis side, in the cross section perpendicular to the center axis of the sheath section 12, are substantially equal to the center-axis-side outer shapes of the air feed extension portion 96a, water feed extension portion 96b and channel extension portion 96c of the air feed inner tube 47a, water feed inner tube 47b and channel inner tube 47c, and are disposed at substantially corresponding positions. Thus, the air feed guide portion 94a, water feed guide portion 94b and channel guide portion 94c are inserted into the corresponding air feed tube reception portion 64a, water feed tube reception portion 64b and channel tube reception portion 64c of the node ring 27.

By inserting the tube reception portions 64a, 64b and 64c into the guide portions 94a, 94b and 94c, the bending section 26 can be inserted into the insertion hole 92. Accordingly, when the bending section 26 is inserted into the insertion hole 92, the air feed guide portion 94a, water feed guide portion 94b and channel guide portion 94c guide the air feed tube reception portion 64a, water feed tube reception portion 64b and channel tube reception portion 64c to the air feed extension portion 96a, water feed extension portion 96b and channel extension portion 96c.

By projecting the built-in section 13 from the distal end of the insertion portion 91, the built-in section 13 is transferred to the air feed inner tube 47a, water feed inner tube 47b and channel inner tube 47c in the flexible tube section 53. Since tensile force is applied to the respective inner tubes (built-in tubes) as described above, the built-in section 13 can be guided into the flexible tube section 53 of the sheath section 12. Specifically, the air feed extension portion 96a, water feed extension portion 96b and channel extension portion 96c are received and engaged in the air feed tube reception portion 64a, water feed tube reception portion 64b and channel tube reception portion 64c, and the built-in section 13 can be guided into the sheath section 12 by the air feed extension portion 96a, water feed extension portion 96b and channel extension portion 96c.

Next, the method of using the separation-type endoscope according to the present embodiment is described. In the case of using the separation-type endoscope, as shown in FIG. 2, the built-in section (insertion section) 13 of the main body section 11 is inserted into the sheath section 12, and the sheath section 12 and the main body section 11 are assembled. At this time, as shown in FIG. 8A, the tube coupling reception portion 62 is pulled to the proximal end side (proximal side), relative to the sheath coupling reception portion 56 of the sheath section 12. As a result, since the respective inner tubes 47a, 47b and 47c are pulled to the proximal end side by the tube coupling reception portion 62, the slack in the inner tubes 47a, 47b and 47c is eliminated, as shown in FIG. 8A, and the inner tubes 47a, 47b and 47c can be aligned in predetermined positions. In addition, in the bending tube 51 and tube body 54, the inner tubes 47a, 47b and 47c can be aligned in predetermined positions in a straight state with tension.

Subsequently, the built-in section 13 is inserted into the sheath section 12 from the proximal-end opening of the insertion hole 92 by aligning the air feed tube reception portion 64a, water feed tube reception portion 64b and channel tube reception portion 64c of the bending section 26 of the main body section 11 with the air feed guide portion 94a, water feed guide portion 94b and channel guide portion 94c of the insertion portion 91 of the tube coupling reception portion 62. At this time, since the air feed tube reception portion 64a, water feed tube reception portion 64b and channel tube reception portion 64c of the bending section 26 are engaged with the air feed guide portion 94a, water feed guide portion 94b and channel guide portion 94c of the insertion hole 92, the air feed tube reception portion 64a, water feed tube reception portion 64b and channel tube reception portion 64c are guided toward the air feed extension portion 96a, water feed extension portion 96b and channel extension portion 96c, respectively. If the built-in section 13 is led out forward from the distal-end opening of the insertion hole 92, the air feed extension portion 96a, water feed extension portion 96b and channel extension portion 96c are directly received in the air feed tube reception portion 64a, water feed tube reception portion 64b and channel tube reception portion 64c. In other words, the respective tube reception portions 64a, 64b and 64c of the bending section 26 are guided by the extension portions 96a, 96b and 96c. Further, the built-in section 13 is guided by the inner tubes 47a, 47b and 47c which are aligned in predetermined positions in the straight state with tension. Accordingly, the built-in section 13 is inserted in a predetermined attitude into the tube body 54 and bending tube 51 of the sheath section 12. At this time, since the inner tubes, which serve as guides, are aligned in the predetermined positions in the straight state with tension, these built-in tubes are not entangled. Moreover, the inserted built-in section 13 is not entangled with the built-in tubes. Therefore, even the built-in components, which are inserted in a dense state into the narrow sheath section 12, can smoothly and easily be inserted to a predetermined position.

If the observation unit 23 of the built-in section 13 is positioned at the sheath distal end portion 43 of the sheath section 12 and the attachment portion 67 of the operation section 14 of the main body section 11 is inserted in the attachment reception portion 66 of the tube coupling reception portion 62, the engagement portion 68 of the tube coupling reception portion 62 is engaged with the engagement reception portion 69 of the operation section 14 and the sheath section 12 is locked to the main body section 11.

At last, the tube coupling reception portion 62 is pushed to the distal end side, relative to the sheath coupling reception portion 56, and is restored to the use state shown in FIG. 8B. As a result, slack occurs in the respective inner tubes 47a, 47b and 47c, and when the bending section 26 is bent, the advancement/retreat of the inner tubes 47a, 47b and 47c is facilitated. Since the amount of bending force is not increased by stretching the inner tubes, the bending operation becomes easier.

The outer tubes 63a, 63b and 63c, which extend from the tube coupling reception portion 62, are bundled and fixed to the proximal end portion of the operation section 14, and the outer tubes 63a, 63b and 63c are connected to a peripheral device. By using the separation-type endoscope, the preparation for which has been completed, observation of a body cavity, etc. can be performed.

After the use of the separation-type endoscope is finished, the endoscope cover is detached. Specifically, the separation button 71 of the operation section 14 is operated, and the sheath section 12 is unlocked from the main body section 11. Then, the built-in section 13 of the main body section 11 is drawn out of the sheath section 12. Thereafter, the sheath section (endoscope cover) is discarded.

With the above-described structure, the following advantageous effects can be obtained. Specifically, in the separation-type endoscope of the present embodiment, by moving the tube coupling reception section 62 to the proximal end side, relative to the sheath coupling reception portion 56, the respective inner tubes 47a, 47b and 47c are stretched and the slack in the inner tubes 47a, 47b and 47c is eliminated. At this time, since the extension portions 96a, 96b and 96c are disposed at predetermined positions in the bending tube 51 and tube body 54, the built-in section 13 can be guided by the parts of the inner tubes 47a, 47b and 47c. Furthermore, since the extension portions 96a, 96b and 96c and the tube reception portions 64a, 64b and 64c are exactly aligned, the insertion of the built-in section 13 can be made easier.

In particular, in the separation-type endoscope of the present embodiment, the manifold portion 72 is formed in the vicinity of the insertion portion 91 in the tube coupling reception portion 62. Since the proximal end portions of the inner tubes 47a, 47b and 47c are connected and fixed to the connection ports 78a, 78b and 78c of the manifold portion 72, the extension portions 96a, 96b and 96c, which are continuous with the outwardly displaced lead-out portions 97a, 97b and 97c of the inner tubes 47a, 47b and 47c, are disposed on the distal side of the insertion portion 91. Therefore, since the tube reception portions 64a, 64b and 64c can be positioned and guided to the extension portions 96a, 96b and 96c which are disposed on the distal side of the insertion portion 91, the built-in section 13 can easily be inserted into the tube body 54 and bending tube 51.

The tube reception portions 64a, 64b and 64c are formed so as to correspond to the respective inner tubes 47a, 47b and 47c. The guide portions 94a, 94b and 94c are formed so as to correspond to the tube reception portions 64a, 64b and 64c. In addition, the outer shapes of the guide portions 94a, 94b and 94c on the center axis side thereof are substantially equal to the center-axis-side outer shapes of the respective extension portions 96a, 96b and 96c. Therefore, the tube reception portions 64a, 64b and 64c can smoothly be guided continuously to the extension portions 96a, 96b and 96c of the inner tubes 47a, 47b and 47c.

Next, a description is given of a cover-type endoscope according to a second embodiment of the present invention. The basic structure of the cover-type endoscope according to this second embodiment is the same as that of the cover-type endoscope according the above-described embodiment. In the second embodiment, an elastic member 101 is provided between the sheath coupling reception portion 56 and the tube coupling reception portion 62. By the elastic force of the elastic member 101, a predetermined tension is applied to the built-in tubes.

The elastic member, in this example, is a coil-like elastic member 101. The elastic member 101 is wounded and fitted on the outer periphery of the slide reception portion 87. Further, the elastic member 101 is interposed between the rear end of the sheath coupling reception portion 56 and the distal end surface of the block portion 77 of the tube coupling reception portion 62. Accordingly, the elastic member 101 urges the tube coupling reception portion 62 to the proximal side, relative to the sheath coupling reception portion 56, and continuously applies a predetermined tension to the built-in tubes. Thus, there is no slack in the built-in tubes, and the built-in tubes are not entangled. In addition, the built-in tubes are not entangled with the built-in section 13 that is inserted, and the function of guiding the built-in section can be exhibited. Therefore, even the built-in components, which are inserted in a dense state into the narrow sheath section 12, can smoothly be inserted to a predetermined position.

Since the elastic member 101 is elastic urging means, the built-in tubes are advancible/retreatable. Therefore, the movement of the built-in tubes is not hindered at the time of bending, and the bending operation is easy. Moreover, the operation of pushing the tube coupling reception portion is unnecessary, and the handling operation is easy.

The elastic member 101 may be, for instance, a plate spring member or a bellows-like member. Any kind of elastic member is usable if it can elastically urge the tube coupling reception portion, relative to the sheath coupling reception portion. Besides, the elastic member is not limited to a type which is interposed between members, and may be a type which pulls members.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope cover which is detachably fitted on an insertion section of an endoscope, comprising:
a cylindrical sheath main body which is detachably fitted on the insertion section of the endoscope;
an operation section coupling member which is provided at a proximal end portion of the sheath main body and is capable of being coupled to an operation section of the endoscope; and
a built-in tube which is disposed in the sheath main body and has a distal end attached to a distal end portion of the sheath main body,
wherein the operation section coupling member includes a sheath main body support member to which a proximal end of the sheath main body is connected, and a built-in tube support member to which a proximal side portion of the built-in tube is connected, and
the built-in tube support member is coupled to the sheath main body support member in a manner to be movable relative to the sheath main body support member in a longitudinal axial direction of the sheath main body.

2. The endoscope cover according to claim 1, wherein one of the sheath main body support member and the built-in tube support member is provided with a cylindrical slide portion which extends in the longitudinal axial direction of the sheath main body,
the other of the sheath main body support member and the built-in tube support member is provided with a cylindrical slide reception portion which is slidably fitted on the slide portion, and
the slide portion and the slide reception portion are relatively movable in the longitudinal axial direction, thereby to adjust slack in the built-in tube.

3. The endoscope cover according to claim 1, wherein an elastic member, which elastically urges the built-in tube support member toward the operation section side, relative to the sheath main body support member, is provided between the sheath main body support member and the built-in tube support member.

4. The endoscope cover according to claim 1, wherein the built-in tube support member includes an inlet for insertion of the insertion section of the endoscope, and a guide portion which restricts a direction of insertion of the insertion section of the endoscope, which is inserted from the inlet into the endoscope cover, and guides the insertion section of the endoscope to a position where slack in the built-in tube is eliminated.

5. A cover-type endoscope in which an endoscope cover is detachably fitted on an insertion section of the endoscope, the endoscope cover comprising:
a cylindrical sheath main body which is detachably fitted on the insertion section of the endoscope;
an operation section coupling member which is provided at a proximal end portion of the sheath main body and is capable of being coupled to an operation section of the endoscope; and
a built-in tube which is disposed in the sheath main body and has a distal end attached to a distal end portion of the sheath main body,
wherein the operation section coupling member includes a sheath main body support member to which a proximal end of the sheath main body is connected, and a built-in tube support member to which a proximal side portion of the built-in tube is connected, and
the built-in tube support member is coupled to the sheath main body support member in a manner to be movable relative to the sheath main body support member in a longitudinal axial direction of the sheath main body.
